(19) United States
(12) Patent Application Publication
(10) Pub. No.: US 2023/0347063 A1
(43) Pub. Date: Nov. 2, 2023

(54) CONTAINER WITH PLUNGER AND LOCKING ARRANGEMENT

(71) Applicant: SCHOTT Pharma Schweiz AG, St. Gallen (CH)

(72) Inventor: Raymond Moser, Engelburg (CH)

(73) Assignee: SCHOTT Pharma Schweiz AG, St. Gallen (CH)

(21) Appl. No.: 18/307,417

(22) Filed: Apr. 26, 2023

(30) Foreign Application Priority Data
Apr. 29, 2022 (EP) 22170978.5

(51) Int. Cl. A61M 5/315

(52) U.S. Cl. CPC A61M 5/31501; A61M 2005/3151; A61M 2005/31508; A61M 2205/0216

(57) ABSTRACT

A syringe includes: a container body including an inner wall; a plunger including a plunger rod, the plunger rod being disposed at least partially within the container body; and a locking arrangement configured for locking a position of the plunger rod relative to the container body and for being operable between at least a locked position and an unlocked position, the locking arrangement including at least one deformable element which is configured for applying a locking force between the plunger rod and the inner wall of the container body when the locking arrangement is in the locked position and for releasing the locking force between the plunger rod and the inner wall of the container body when the locking arrangement is changed from the locked position to the unlocked position.

### CROSS REFERENCE TO RELATED APPLICATIONS

This claims priority to European patent application no. EP 22170978.5, entitled “CONTAINER WITH PLUNGER AND LOCKING ARRANGEMENT FOR THE PLUNGER”, filed Apr. 29, 2022, which is incorporated herein by reference.

### BACKGROUND OF THE INVENTION

### 1. Field of the Invention

The application relates to a container, in particular a syringe or a cartridge, including a container body, a plunger and a locking arrangement.

### 2. Description of the Related Art

Medical syringes, in particular when filled with a pharmaceutical and stored at low temperatures, often show large motion of the plunger during the cooling and thawing processes. This motion is mainly due to thermal expansion and contraction of the content of the syringe during the temperature changes and a resultant pressure difference between an inside of the syringe and the environment.

Plunger motion poses a risk to the sterility of the syringe content. For example, a travel length of the plunger due to plunger motion can exceed a tolerance limit of the stopper, which typically forms the distal end of the plunger and which, often having a plurality of elastic sealing ribs, provides a certain tolerance regarding plunger motion without loss of sterility. Sterility of the syringe content is assumed to be lost when a position on the inner wall of the syringe which lies outside the volume enclosed by the stopper in one situation occurs to be enclosed by the stopper, or to lie in a region within a length of the stopper that is considered to be not safely sealed, in another situation, and vice versa. As a consequence, inappropriate choice of the plunger may result in a loss of sterility in later applications.

Choosing a plunger in accordance with the tolerance characteristics dependent on temperature requirements of an intended content of a syringe has proven inconvenient in many situations of supply and production. Furthermore, glass syringes often do not meet the requirements of container closure integrity, CCI, at very low temperatures, for example, around minus 80 degrees Celsius, which are typical storage temperatures of various vaccines.

Therefore, what is needed in the art is a technique that avoids, or at least mitigates, at least one the aforesaid disadvantages.

### SUMMARY OF THE INVENTION

According to a first aspect, a container, in particular a syringe or a cartridge, is provided. The container includes a container body, a plunger including a plunger rod which is disposed at least partially within the container body, and a locking arrangement for locking a position of the plunger rod relative to the container body. The locking arrangement is operable between at least a locked position and an unlocked position. The locking arrangement includes at least one deformable element which is configured for applying a locking force between the plunger rod and an inner wall of the container body when the locking arrangement is in the locked position and for releasing a locking force between the plunger rod and the inner wall of the container body when the locking arrangement is changed from the locked position to the unlocked position.

Thus, plunger motion can be effectively reduced. Moreover, a tightness of the container due to a sealing between the plunger rod and the inner wall of the container can be enhanced. Furthermore, changing the locking arrangement from the locked position to the unlocked position can be reversible. In other words, the locking arrangement can be operable repeatedly. In addition, a force which is to be applied in an operating direction of the plunger rod and which is required for moving the plunger rod when the locking arrangement is in the locked position can be adjusted by suitable adjustment of the locking force.

The locking arrangement can be at least essentially attached to the plunger rod. In addition, parts of the locking arrangement can be formed integrally with the plunger rod.

The deformable element can be arranged to extend along an inner periphery of the container body. The inner periphery of the container body can be essentially circular, and the deformable element can have an essentially circular shape. As an alternative, the inner periphery of the container body can be essentially polygonal and the deformable element can have an essentially polygonal shape. As another alternative, the inner periphery of the container body can be essentially oval and the deformable element can have an essentially oval shape. The deformable element can further extend adjacent the inner wall of the container body in a longitudinal, in particular an axial, direction and/or in an angular direction of the container body.

The deformable element can be further configured to seal a gap between the inner wall of the container body and the plunger rod when the locking arrangement is in the locked position.

The locking arrangement can further include a lateral protuberance which extends from a portion of the plunger rod. In addition, the deformable element can be arranged to be pressed towards the lateral protuberance when the locking arrangement is in the locked position.

The locking arrangement can further include a sliding member which is movable relative to the plunger rod and configured to apply a pressing force on the deformable element towards the lateral protuberance when the locking arrangement is in the locked position.

The lateral protuberance and/or the sliding member can be configured to cause deformation and/or displacement of at least a portion of the deformable element towards the inner wall of the container when the locking arrangement is in the locked position. The lateral protuberance can extend from the plunger rod. In addition, the sliding member can include a tube in which the plunger rod extends at least partially.

The locking arrangement can further include at least one actuating member configured for moving the sliding member towards the deformable element when bringing the locking arrangement to the locked position and/or for releasably holding the locking arrangement in the locked position. The actuating member can include at least one wedge.

The actuating member can be arranged between the sliding member and a thumb rest of the plunger rod. The actuating member can be at least partially formed integrally with the sliding member at a proximal end of the sliding member facing a thumb rest of the plunger rod. In addition or as an alternative, the actuating member can be at least partially formed integrally with the thumb rest of the plunger rod, the thumb rest being movable relative to the lateral protuberance.

The locking arrangement can include a plurality of actuating members which are usable interchangeably, wherein at least two of the plurality of actuating members differ with respect to a distance by which the sliding member is moved relative to the plunger rod when the actuating member is used and operated, respectively. The plurality of actuating members can include a plurality of pins, wherein at least two of the pins differ with respect to a thickness. The plurality of actuating members can be usable interchangeably for adjusting a total break-loose force and/or a total gliding force of the plunger. The total break-loose force and the total gliding force of the plunger can include a break-loose force and a gliding force of a stopper attached to the plunger rod, respectively, plus a locking force produced by way of the locking arrangement.

The container may include polymer and/or glass. The container can be uncoated or silicone-free coated, in particular at an inner surface of the container, more particularly in an area contacting with the deformable element when the locking arrangement is in the locked position. A material of the deformable element can include a silicone. As an alternative, the deformable element can be silicone-free, in particular including rubber. In addition, or as an alternative, the deformable element can be silicone oil-free coated, optionally liquid-free coated, or, optionally, can be uncoated. The plunger can include a coating with polytetrafluorethylene, PTFE. The container, including the container body, the plunger and the locking arrangement, can be silicone-free.

The container can be a pharmaceutical container, in particular a syringe or a cartridge.

The container can be pre-filled.

The deformable element can be configured to seal the gap between the inner wall of the container body and the plunger rod against a pressure difference of at least 500 millibars, optionally at least 600 millibars, optionally at least 800 millibars, at a temperature of minus 80 degrees Celsius.

The locking arrangement can be configured to reduce a motion of the plunger rod relative to the container body to less than 3 millimeters, optionally less than 2 millimeters, optionally less than 1.5 millimeters, when applying a pressure difference of 800 millibars at a temperature of minus 80 degrees Celsius.

The deformable element can have a Shore hardness in the range from 25 to 80 Shores A, optionally in the range from 25 to 50 Shores A, optionally in the range from 30 to 35 Shores A.

The locking arrangement can be configured to apply a locking force in the range from 5 to 15 newtons. The locking force can be defined as a force which is to be applied in an operating direction of the plunger rod and which is required for moving the plunger rod, adjusted for other forces, in particular a break-loose force and/or a gliding force of a stopper attached to the plunger rod.

In particular, the container can be a syringe with a brimful volume in the range from 0.8 to 2 milliliters, a break-loose force of a stopper attached to the plunger rod can be less than 12 newtons, a gliding force of the stopper can be less than 7 newtons, and a locking force of the locking arrangement can be in the range from 12 to 25 newtons. As an alternative, the container can be a syringe with a brimful volume in the range from 8 to 20 milliliters, a break-loose force of a stopper attached to the plunger rod can be less than 35 newtons, a gliding force of the stopper can be less than 20 newtons, and a locking force of the locking arrangement can be in the range from 20 to 30 newtons. As another alternative, the container can be a syringe with a brimful volume in the range from 30 to 60 milliliters, a break-loose force of a stopper attached to the plunger rod can be less than 45 newtons, a gliding force of the stopper can be less than 25 newtons, and a locking force of the locking arrangement can be in the range from 30 to 45 newtons.

A contact length between the deformable element and the inner wall of the container body can be in the range from 0 to 1 millimeters, optionally 0 millimeters, when the locking arrangement is in the unlocked position. In addition, or as an alternative, a contact length between the deformable element and the inner wall of the container body can be in the range from 2 to 20 millimeters, optionally in the range from 3 to 4 millimeters, when the locking arrangement is in the locked position.

The deformable element can have a single annular contact area with the inner wall of the container body when the locking arrangement is in the locked position.

A contact area of the deformable element with respect to the inner wall of the container body can be larger when the locking arrangement is in the locked position than when the locking arrangement is in the unlocked position.

The container body can be transparent in the region of a contact area of the deformable element with respect to the inner wall of the container body. In addition, a surface of the deformable element as seen through the container body can appear darker when in contact with the contact area than when in a non-contacting state.

The plunger can further include a stopper attached to the plunger rod. A material of the stopper and a material of the deformable element can be not the same. The material of the stopper can include a rubber, optionally a brombutyl rubber or ethylene propylene diene monomer, EPDM, rubber.

The deformable element can be not in direct contact with the stopper. In this case, the lateral protrusion can extend between the deformable element and the stopper. As an alternative, the deformable element can be in direct contact with the stopper. In this case, the stopper, in particular a proximal portion of the stopper, can at least partially constitute the lateral protrusion.

The following parameters can be fulfilled while the locking arrangement is in the locked position: the brimful volume of the container body is 0.5 to 100 milliliters, optionally 1 to 20 milliliters, optionally 1 to 5 milliliters; the container is prefilled with a liquid, optionally with 10% to 90%, optionally 20% to 80%, optionally 30% to 70%, optionally 40% to 60% of the brimful volume of the container, optionally the liquid additionally including a sugar or salt; the container includes a headspace of gas, optionally air, of 0 to 10 millimeters, optionally 0.5 to 5 millimeters, optionally 1 to 4 millimeters, optionally 1.5 to 3 millimeters. In addition, the plunger rod movement can be 3 millimeters or less, optionally 2 millimeters or less, optionally 1 millimeter or less, optionally 0.5 millimeters or less, optionally 0 millimeters, after one or both of the following test conditions were applied: the pressure outside the container with regard to the inside was reduced by 100 to 1000 millibars, optionally 200 to 900 millibars, optionally 300 to 800 millibars, optionally 400 to 600 millibars, optionally 430 millibars for 1 second to 1 week, optionally 1 minute to 24 hours, optionally 1 hour to 6 hours, optionally 180 minutes, or the container was stored at −210° C. to 25° C., optionally −120° C. to 0° C., optionally −100° C. to −15° C., optionally −90° C. to −40° C., optionally −80° C., for 1 second to 2 years, optionally 1 minute to 7 days, optionally 30 minutes to 24 hours, optionally 1 hour to 6 hours, optionally 180 minutes.

### Optional Embodiments

The embodiments according to the following items are optional:

Item 1. Container (100; 700), in particular a syringe or a cartridge, including:
a container body (110);
a plunger (130; 530; 630) including a plunger rod (132; 332; 432; 532; 632), the plunger rod (132; 332; 432; 532; 632) disposed at least partially within the container body (110); and
a locking arrangement (150; 350; 450; 550; 650) for locking a position of the plunger rod (132; 332; 432; 532; 632) relative to the container body (110),
characterized in that
the locking arrangement (150; 350; 450; 550; 650) is operable between at least a locked position and an unlocked position, wherein the locking arrangement (150; 350; 450; 550; 650) includes at least one deformable element (152; 352; 452) which is configured for applying a locking force between the plunger rod (132; 332; 432; 532; 632) and an inner wall (112) of the container body (110) when the locking arrangement (150; 350; 450; 550; 650) is in the locked position and for releasing a locking force between the plunger rod (132; 332; 432; 532; 632) and the inner wall (112) of the container body (110) when the locking arrangement (150; 350; 450; 550; 650) is changed from the locked position to the unlocked position.

Item 2. Container according to item 1, wherein the locking arrangement (150; 350; 450; 550; 650) is attached to the plunger rod (132; 332; 432; 532; 632).

Item 3. Container according to item 1 or 2, wherein the deformable element (152; 352; 452) is arranged to extend along an inner periphery of the container body (110).

Item 4. Container according to item 3, wherein the inner periphery of the container body (110) is essentially circular, and the deformable element (152; 352; 452) has an essentially circular shape, or wherein the inner periphery of the container body (110) is essentially polygonal and the deformable element (152; 352; 452) has an essentially polygonal shape.

Item 5. Container according to any one of the preceding items, wherein the deformable element (152; 352; 452) is further configured to seal a gap between the inner wall of the container body (110) and the plunger rod (132; 332; 432; 532; 632) when the locking arrangement (150; 350; 450; 550; 650) is in the locked position.

Item 6. Container according to any one of the preceding items, wherein the locking arrangement (150; 350; 450; 550; 650) further includes a lateral protuberance (160; 360; 460) which extends from a portion of the plunger rod (132; 332; 432; 532; 632), and wherein the deformable element (152; 352; 452) is arranged to be pressed towards the lateral protuberance (160; 360; 460) when the locking arrangement (150; 350; 450; 550; 650) is in the locked position.

Item 7. Container according to item 6, wherein the locking arrangement (150; 350; 450; 550; 650) further includes a sliding member (154; 354; 554; 654) which is movable relative to the plunger rod (132; 332; 432; 532; 632) and configured to apply a pressing force on the deformable element (152; 352; 452) towards the lateral protuberance (160; 360; 460) when the locking arrangement (150; 350; 450; 550; 650) is in the locked position.

Item 8. Container according to any one of items 6 or 7, wherein the lateral protuberance (160; 360; 460) and/or the sliding member (154; 354; 554; 654) are configured to cause deformation and/or displacement of at least a portion of the deformable element (152; 352; 452) towards the inner wall (112) of the container (110) when the locking arrangement (150; 350; 450; 550; 650) is in the locked position.

Item 9. Container according to any one of items 6 to 8, wherein the lateral protuberance (160; 360; 460) extends from the plunger rod (132; 332; 432; 532; 632), and the sliding member (154; 354; 554; 654) includes a tube in which the plunger rod (132; 332; 432; 532; 632) extends at least partially.

Item 10. Container according to any one of items 7 to 9, wherein the locking arrangement (150; 350; 450; 550; 650) further includes at least one actuating member (170; 570; 670) configured for moving the sliding member (154; 354; 554; 654) towards the deformable element (152; 352; 452) when bringing the locking arrangement (150; 350; 450; 550; 650) to the locked position and/or for releasably holding the locking arrangement (150; 350; 450; 550; 650) in the locked position.

Item 11. Container according to item 10, wherein the at least one actuating member (170; 570) includes at least one wedge (172; 558, 572).

Item 12. Container according to item 10 or item 11, wherein the actuating member (170; 570; 670) is arranged between the sliding member (154; 354; 554; 654) and a thumb rest (138) of the plunger rod (132; 332; 432; 532; 632).

Item 13. Container according to item 10 or item 11, wherein the at least one actuating member (558, 570; 658, 670) is at least partially formed integrally with the sliding member (554; 654) at a proximal end (558; 658) of the sliding member (554; 654) facing a thumb rest (538; 638) of the plunger rod (532; 632), and/or wherein the actuating member (570, 572; 670, 672) is at least partially formed integrally with the thumb rest (538; 638) of the plunger rod (532; 632), the thumb rest (538; 638) optionally being movable relative to the lateral protuberance (160; 360; 460).

Item 14. Container according to any one of the preceding items, wherein the container body (110) includes polymer and/or glass.

Item 15. Container according to any one of the preceding items, wherein the container body (110) is uncoated or silicone-free coated in an area contacting with the deformable element (152; 352; 452) when the locking arrangement (150; 350; 450; 550; 650) is in the locked position.

Item 16. Container according to any one of the preceding items, wherein a material of the deformable element (152; 352; 452) includes a silicone, and/or wherein the deformable element (152; 352; 452) is silicone oil-free coated, optionally liquid-free coated, or, optionally, is uncoated.

Item 17. Container according to any one of the preceding items, wherein the container (100; 700) is a pharmaceutical container, in particular a syringe or a cartridge.

Item 18. Container according to any one of the preceding items, wherein the container (700) is pre-filled.

Item 19. Container according to any one of the preceding items in combination with item 5, wherein the deformable element (152; 352; 452) is configured to seal the gap between the inner wall of the container body (110) and the plunger rod (132; 332; 432; 532; 632) against a pressure difference of at least 500 millibars, optionally at least 600 millibars, optionally at least 800 millibars, at a temperature of minus 80 degrees Celsius.

Item 20. Container according to any one of the preceding items, wherein the locking arrangement (150) is configured to reduce a motion of the plunger (130) relative to the container body (110) to less than 3 millimeters, optionally less than 2 millimeters, optionally less than 1.5 millimeters, optionally less than 0.5 millimeters, when applying a pressure difference of 500 millibars, optionally 600 millibars, optionally 800 millibars, at a temperature of minus 20 degrees Celsius, optionally minus 50 degrees Celsius, optionally minus 80 degrees Celsius.

Item 21. Container according to any one of the preceding items, wherein the deformable element (152; 352; 452) has a Shore hardness in the range from 25 to 80 Shores A, optionally in the range from 25 to 50 Shores A, optionally in the range from 30 to 35 Shores A.

Item 22. Container according to any one of the preceding items, wherein the locking arrangement (150; 350; 450; 550; 650) is configured to apply a locking force in the range from 5 to 15 newtons.

Item 23. Container according to any one of the preceding items, wherein a contact length (L_C) between the deformable element (152; 352; 452) and the inner wall (112) of the container body (110) is in the range from 0 to 1 millimeters, optionally 0 millimeters, when the locking arrangement (150; 350; 450; 550; 650) is in the unlocked position and/or wherein a contact length (L_C) between the deformable element (152; 352; 452) and the inner wall (112) of the container body (110) is in the range from 2 to 20 millimeters, optionally in the range from 3 to 4 millimeters, when the locking arrangement (150; 350; 450; 550; 650) is in the locked position.

Item 24. Container according to any one of the preceding items, wherein the deformable element (152; 352; 452) has a single annular contact area with the inner wall (112) of the container body (110) when the locking arrangement (150; 350; 450; 550; 650) is in the locked position.

Item 25. Container according to any one of the preceding items, wherein a contact area of the deformable element (152; 352; 452) and the inner wall (112) of the container body (110) when the locking arrangement (150; 350; 450; 550; 650) is in the locked position is larger than when the locking arrangement (150; 350; 450; 550; 650) is in the unlocked position.

Item 26. Container according to any one of the preceding items, wherein the container body (110) is transparent in the region of a contact area of the deformable element (152; 352; 452) and the inner wall (112) of the container body (110), and a surface of the deformable element (152; 352; 452) as seen through the container body (110) appears darker when in contact with the contact area than when in a non-contact state.

Item 27. Container according to any one of the preceding items, wherein the plunger (130; 530; 630) further includes a stopper (134) attached to the plunger rod (132; 332; 432; 532; 632), wherein optionally a material of the stopper (134) and a material of the deformable element (152; 352; 452) are different.

Item 28. Container according to item 27, wherein the material of the stopper (134) includes a rubber, optionally a brombutyl rubber or ethylene propylene diene monomer, EPDM, rubber.

Item 29. Container according to any one of the preceding items, wherein the deformable element (152; 352; 452) is not in direct contact with the stopper (134).

Item 30. Container according to any one of the preceding items, wherein the locking arrangement (150; 350; 450; 550; 650) is in the locked position and the following parameters are fulfilled:
the brimful volume of the container body (110) is 0.5 to 100 milliliters, optionally 1 to 20 milliliters, optionally 1 to 5 milliliters;
the container (700) is prefilled with a liquid (710), optionally with 10% to 90%, optionally 20% to 80%, optionally 30% to 70%, optionally 40% to 60% of the brimful volume of the container (700), optionally wherein the liquid (710) includes a sugar or salt,
the container (700) includes a headspace of gas (712), optionally air, of 0 to 40 millimeters, optionally 0.5 to 5 millimeters, optionally 1 to 4 millimeters, optionally 1.5 to 3 millimeters;
wherein a movement of the plunger rod is 3 millimeters or less, optionally 2 millimeters or less, optionally 1 millimeter or less, optionally 0.5 millimeters or less, optionally 0 millimeters, after one or both of the following test conditions were applied:
the pressure outside the container (700) with regard to the inside was reduced by 100 to 1000 millibars, optionally 200 to 900 millibars, optionally 300 to 800 millibars, optionally 400 to 600 millibars, optionally 430 millibars for 1 second to 1 week, optionally 1 minute to 24 hours, optionally 1 hour to 6 hours, optionally 180 minutes;
the container (700) was stored at −210° C. to 25° C., optionally −120° C. to 0° C., optionally −100° C. to −15° C., optionally −90° C. to −40° C., optionally −80° C., for 1 second to 7 days, optionally 1 minute to 24 hours, optionally 1 hour to 6 hours, optionally 180 minutes.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a container according to an example;

FIGS. 2A and 2B are detailed views of the locking arrangement of the container shown in FIG. 1 in different states;

FIGS. 3 and 4 are detailed views of locking arrangements according to further examples;

FIGS. 5 to 6B (that is, FIGS. 5(i), 5(ii), 6A(i), 6A(ii), 6A(iii), 6B(i), 6B(ii), and 6B(iii)) are detailed views of locking arrangements according to further examples in different states;

FIG. 7 is a container according to another example; and

FIG. 8 is a set of different actuating members.

Corresponding reference characters indicate corresponding parts throughout the several views. The exemplifications set out herein illustrate embodiments of the invention, and such exemplifications are not to be construed as limiting the scope of the invention in any manner.

### DETAILED DESCRIPTION OF THE INVENTION

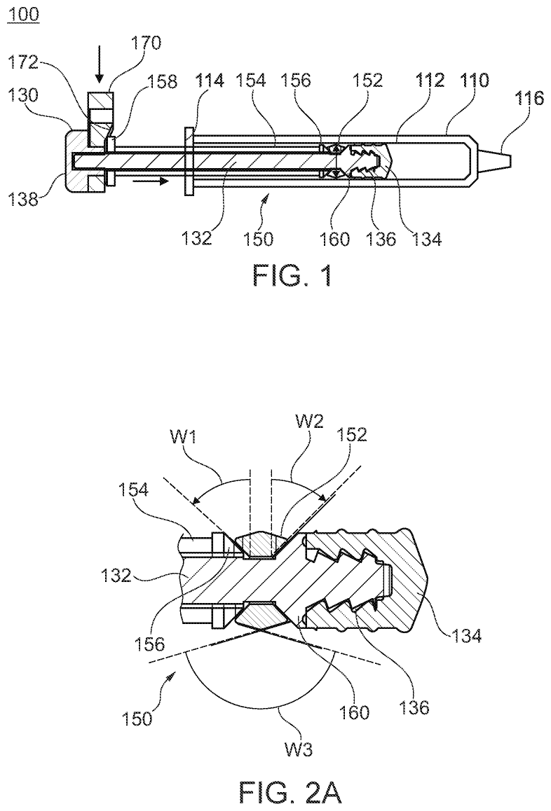

FIG. 1 shows schematically and exemplarily a container 100, in particular a syringe. The container 100 includes a container body 110 and a plunger 130 which extends partially in the container 110 through an opening 114 at a proximal end of the container 110. The plunger 130 is movable in the container body 110, for example, in order to deliver a content of the container body 110 through a tip 116 of the container body 110.

The plunger 130 includes a plunger rod 132 and a stopper 134 which is arranged at a distal end of the plunger rod 13. The stopper 134 delimits a variable volume inside the container 110. To this end, the stopper 134 extends adjacent an inner wall 112 of the container 110. The volume in the container 110 which is delimited by the stopper 134 is variable according to a position of the stopper 134, which can be modified through manipulation by way of the plunger rod 132. A thumb rest 138 is provided at a proximal end of the plunger 130. The thumb rest 138 facilitates a delivering of content through the tip 116 by pushing the plunger 130 in an inward direction, especially in a single-handed operation of the container 100. In the shown example, the stopper 134 is attached to the plunger rod 132 by way of a thread 136. The thread 136 facilitates the application of a pulling force on the stopper 134 when pulling the plunger rod 132 in an outward direction. In other examples, other types of connections between the stopper 134 and the plunger rod 132 are used. In some examples, the stopper 134 is formed integrally with the plunger rod 132.

With respect to the aforesaid features and functions, the container 100 essentially resembles a conventional syringe, in particular for pharmaceutical and/or medical use. Furthermore, in some examples the container body 110 includes polymer and/or glass.

The container 100 further includes a locking arrangement 150. The locking arrangement 150 is essentially attached to the plunger rod 132 and includes a deformable element 152, a sliding member 154 having a distal end 156 and a proximal end 158, a lateral protrusion 160 and an actuating member 170.

The sliding member 154 is movable relative to the plunger rod 132 parallel to a longitudinal extension of the plunger rod 132. Moving the sliding member 154 in the distal direction, as indicated in FIG. 1 by the arrow parallel to the plunger rod 132, will cause the distal end 156 of the sliding member 154 to exert a pushing force on the deformable element 152. The deformable element 152 in turn abuts the lateral protrusion 160 extending from the plunger rod 132. As an effect, a compression force will be applied to the deformable element 152.

As shown in FIG. 1, both the distal end 156 of the sliding member 154 and the lateral protrusion have a wedge-shaped profile. As further indicated by the double arrow in FIG. 1, the wedge-shaped profiles of the distal end 156 and the lateral protrusion will cause displacement and/or deformation of the deformable element 152 towards the inner wall 112 of the container 110 under the influence of the longitudinal force applied to the sliding member 154.

By displacing and/or deforming the deformable element 152 towards the inner wall 112, a friction between the deformable element 152 and the inner wall 112 is increased. As a result, motion of the plunger 130 relative to the container body 110 in a longitudinal direction is inhibited.

Furthermore, by displacing and/or deforming the deformable element 152 towards the inner wall 112, a gap between the plunger 130, in particular the plunger rod 132, and the inner wall 112 of the container body 110 becomes sealed. As an effect, a tightness of the container 110 is increased.

In the shown example, each of the container body 110, the sliding member 154, the deformable element 152 and the lateral protrusion 160 has, at least essentially, a radially symmetric shape. For example, the container body 110 is in some implementations at least essentially cylindrical. Moreover, in some examples, the sliding member 154 is essentially tube-shaped, and the deformable element 152 and the lateral protrusion 160 are at least essentially circular and/or ring-shaped. In other examples, each of the container body 110, the sliding member 154, the deformable element 152 and the lateral protrusion 160 has, at least essentially, a polygonal cross-section in a plane that is orthogonal to a longitudinal direction of the cylinder 110. A polygonal cross-section of each of these parts corresponds in some examples to a polygon having an odd number of corners.

The deformable element 152 is essentially ring-shaped. Thus, when the distal end 156 of the sliding member 154 moves towards the lateral protrusion 160, an inner diameter of the deformable element 152 becomes expanded. Accordingly, a profile of the deformable element 152 becomes displaced towards the inner wall 112 of the container 110. Furthermore, the deformable element 152 will be pressed against the inner wall 112 of the container 110, resulting in a deformation of the profile of the deformable element 152.

The deformable element 152 is in some examples made of elastic material, such as an elastic silicone. In addition, in some examples the deformable element 152 has a Shore hardness in the range from 25 to 80 Shores A, optionally in the range from 25 to 50 Shores A, optionally in the range from 30 to 35 Shores A.

In addition, in some examples a material of the stopper 134 and the material of the deformable element 152 are different. The material of the stopper 134 includes in some examples a rubber, optionally a brombutyl rubber. Furthermore, in some examples the deformable element 152 and the stopper 134 are not in direct contact.

When a pressing force acting on the deformable element 152, as described above, is released, by moving the sliding member 154 outwardly, the deformable element 152 will return towards its natural state. Accordingly, the deformable element 152 will adopt a state in which it is less displaced towards, and less deformed by, the inner wall 112 of the container body 110. As a consequence, a friction between the deformable element 152 and the inner wall 112 is reduced. A motion of the plunger 130 relative to the container body 110 thus becomes less or not inhibited by the locking arrangement 150. In this manner, the locking arrangement 150 is changed from a locked position to an unlocked position, and vice versa, by moving the sliding member 154 away from, or towards, a position in which the distal end 156 of the sliding member 154 presses on the deformable element 152.

The locking arrangement 150 further includes an actuating member 170. The actuating member 170 is movable between an unlocked and a locked position. Furthermore, the actuating member 170 is configured to hold the sliding member 154 in a position corresponding to the locked position of the locking arrangement 150, when the actuating member 170 is in its locked position, and to release the sliding member 154 from the position corresponding to the locked position of the locking arrangement 150, when the actuating member 170 is moved from its locked position to its unlocked position.

In some examples, the actuating member 170 is further configured to move the sliding member 154 from a position corresponding to an unlocked position of the locking arrangement 150 to a position corresponding to the locked position of the locking arrangement 150, when the actuating member 170 is moved from its unlocked position to its locked position.

In the example shown in FIG. 1, the actuating member 170 is arranged between the thumb rest 138 and the proximal end 158 of the sliding member 154. Furthermore, in the shown example, the actuating member 170 is formed separate from the plunger 130 and the sliding member 154. In some examples, the actuating member 170 is formed as a separate part which is removable from the plunger 130. In other examples, the actuating member 170 is movably attached to the plunger 130.

In some examples, the actuating member 170 further serves as a seal and/or a tampering evidence of the container 100. For this purpose, the actuating member 170, for example, in the form of a spacer, is attached to the plunger 130 in a position corresponding to a locked position of the locking arrangement 150 and is irreversibly removable from the plunger 130.

FIG. 1 shows the locking arrangement 150 in the unlocked position. As can be seen from FIG. 1, moving the sliding member 154 in an inward direction, corresponding to a locked position of the locking arrangement 150, will enable the insertion of the actuating member 170 between the thumb rest 138 and the proximal end 158 of the sliding member 154. Once inserted, the actuating member 170 will keep the locking arrangement 150 releasably in the locked position.

The actuating member 170 includes a wedge 172, which is facing the proximal end 158 of the sliding member 154. By way of the wedge 172, when the actuating member 170 is moved into a position between the thumb rest 138 and the sliding member 154, as indicated by the arrow next to the actuating member 170 in FIG. 1, the sliding member 154 will be moved in the inward direction and, in consequence, the locking arrangement 150 will be brought into the locked position. In this way, the wedge 172 facilitates an operation of the locking system 150.

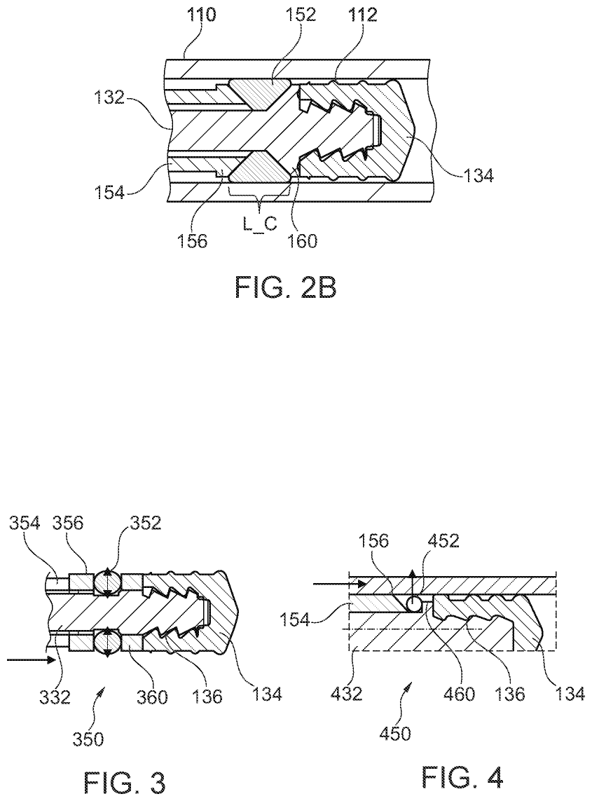

FIGS. 2A and 2B show details of the locking arrangement 150 in the unlocked and in the locked position. Identical reference numbers as in FIG. 1 denote identical features. As shown in FIG. 2A, the distal end 156 of the sliding member 154 is angled relative to an orthogonal plane by an angle W1. Moreover, the surface of the lateral protrusion 160 is angled relative to an orthogonal plane by an angle W2. The angles W1, W2 determine an extent of displacement of the deformable element 152 in relation to a length by which the sliding member 154 is moved towards the lateral protrusion 160. In general, the larger the angles W1, W2 are chosen, the less of an operating force needs to be applied on the sliding member 154, at the expense of an increased length by which the sliding member 154 needs to be moved when operating the locking arrangement 150.

Furthermore, the profile of the deformable element 152 is peaked at an outer surface of the deformable element 152 by the angle W3. A peaked profile causes an increased local locking force in the region of the peak, when the locking arrangement 150 is in the locked position. This gives rise to an increased local friction and improved sealing by the deformable element. At the same time, a contact length between the deformable element 152 and the inner wall 112 of the container 110 when the locking arrangement 150 is in the unlocked position is effectively reduced. Furthermore, the angle W3 determines a deformation of the deformable element 152 when the latter is pressed against the inner wall 112 in the locked position of the locking arrangement 112. The larger the angle W3 is chosen, the larger will be a contact length of the deformable element 112 with the inner wall 112 of the container 110 when the locking arrangement 150 is in the locked position. Moreover, a homogeneity of the contact pressure over the contact length between the deformable element 152 and the inner wall 112 can be adjusted by the choice of the angle W3.

FIG. 2B shows the locking arrangement 150 in the locked position. As described above in connection with FIG. 2A, the outer surface of the deformable element 152 is pressed against the inner wall 112 of the container 110, causing deformation of the profile of the deformable element 152. In particular, the peaked outer surface of the deformable element 152 in the relaxed state has been deformed to create a contact length L_C with the inner wall 112 which extends essentially over a total length of the deformable element 152.

In some examples, the container body 110 is uncoated or silicone-free coated in the area which is in contact with the deformable element 152 when the locking arrangement 150 is in the locked position. In addition, in some examples a material of the deformable element 152 includes a silicone and/or the deformable element 152 is silicone oil-free coated, optionally liquid-free coated, or, optionally, is uncoated.

Furthermore, in some examples, the container body 110 is transparent in the area which is in contact with the deformable element 152 when the locking arrangement 150 is in the locked position. In addition, a surface of the deformable element 152 has one or more characteristics, such as a surface roughness, which alter when the surface of the deformable element 152 is pressed against the inner wall 112 of the container body 110 in such way that an optical appearance of the deformable element 152 as seen from an outside of the container body 110 through the inner wall 112, for example a brightness in a greyscale image, changes. This facilitates an inspection of the locking state of the locking arrangement 150 by a user.

FIG. 3 shows schematically and exemplarily a detail of a locking arrangement 350 according to another example. Identical reference numerals as in the preceding FIGS. 1 to 2B denote identical features. Furthermore, what has been described above regarding the features and functions of the locking arrangement 150 applies analogously to the locking arrangement 350, unless otherwise clear from the following.

In the locking arrangement 350, the lateral protrusion 360 and the distal end 356 of the sliding member 354 are not angled, but they are formed parallel to an orthogonal plane. As an effect, no displacement of the profile of the deformable element 352 towards the inner wall 112 will be caused directly by moving the sliding member 354 towards the lateral protrusion 360, which is attached to the plunger rod 332 adjacent the thread 136. Instead, compression of the profile of the deformable element 352 will occur between the parallel surfaces of the distal end 356 and the lateral protrusion 360. This in turn will lead to a deformation of the profile of the deformable element 352 such that at least an outer portion of the deformable element 352 will be moved towards the inner wall 112, as indicated by the double arrows.

FIG. 4 shows schematically and exemplarily a detail of a locking arrangement 450 according to another example. Identical reference numerals as in the preceding FIGS. 1 to 3 denote identical features. Furthermore, what has been described above regarding features and functions of the locking arrangements 150, 350 applies analogously to the locking arrangement 450, unless otherwise clear from FIG. 4 and the following description.

In the locking arrangement 450, the distal end 156 of the sliding member 154 is angled, whereas the lateral protrusion 460, which extends from the plunger rod 432 adjacent the thread 136, is not angled. Furthermore, a profile of the deformable element 452 is essentially circular.

When the sliding member 154 is moved towards the lateral protrusion 460, the angled distal end 156 will cause a displacement of the profile of the deformable element 452 towards the inner wall 112 of the container 110, as indicated by the arrow next to the deformable element 452. At the same time, the circular shape of the profile of the deformable element 452 facilitates a sliding of the deformable element 452 along the lateral protrusion 460.

In the above examples, a lateral protrusion of each of the locking arrangements is formed separate from the stopper 134. However, in other examples of the described locking arrangements, the lateral protrusion is formed integrally with the stopper 134, especially at a proximal end of the stopper 134. In these examples, firm mounting of the stopper 134 to the plunger rod, for example, by way of a thread, is provided to support a pushing force which is applied to the deformable element when the locking arrangement is in the locked position.

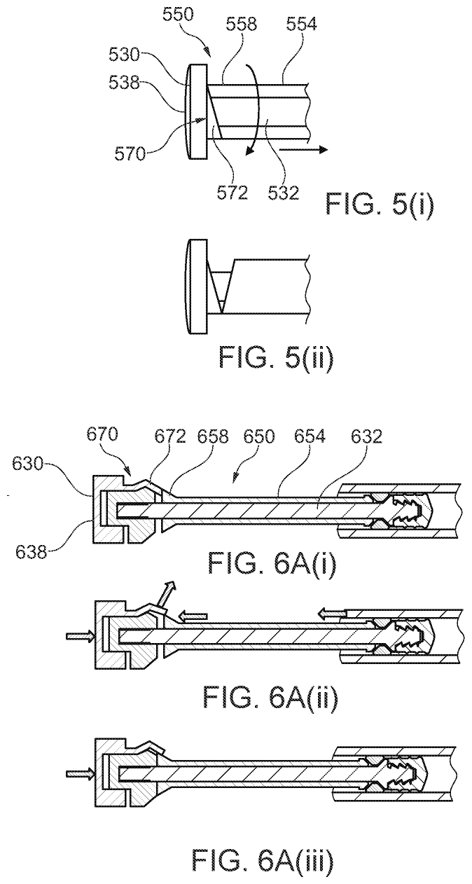

FIG. 5 (which is, FIGS. 5(i) and 5(ii)) shows schematically and exemplarily a detail of a locking arrangement 550 according to another example in an unlocked (upper portion of FIG. 5, that is FIG. 5(i)) and in a locked state (lower portion of FIG. 5, that is, FIG. 5(ii)). What has been described above regarding features and functions of the locking arrangements 150, 350, 450 applies analogously to the locking arrangement 550, unless otherwise clear from FIG. 5 and the following description.

In the locking arrangement 550, the at least one actuating member 570 includes a pair of mutually engaging wedges 558, 572, one of which is integrally formed with the sliding member 554 to constitute a proximal end 558 of the sliding member 554, and the other one is formed at an underside of the thumb rest 538 of the plunger 530.

As indicated in the upper portion of FIG. 5 (that is, FIG. 5(i)) by the curved arrow, the sliding member 554 can be rotated relative to the plunger rod 532 around its longitudinal axis. The relative position of the wedges 558, 572 is changed in this way, resulting in a motion of the sliding member 554 parallel to its longitudinal axis, i.e., towards or away from the lateral protrusion (not shown) of the locking arrangement 550.

As shown in the lower portion of FIG. 5 (that is, FIG. 5(ii)), a locked position of the locking arrangement 550 corresponds in some examples to a symmetric position of the wedges 558, 572. In this position, no torque on the sliding member 554 results from a reaction force which is exerted on the sliding member 554 by the deformable element (not shown). In addition, a surface friction between the wedges 558, 572 is sufficient in some examples to hold the locking arrangement 550 in the locked position.

In the shown example, the sliding member 554 is rotatable relative to the plunger rod 532 and the thumb rest 538. In other examples, the wedge 572, in some examples together with the thumb rest 538, is rotatable relative to the sliding member 554 and the plunger rod 532.

In a modified example of the locking arrangement 550, the actuating member 570 includes a pair of engaging threads in place of the wedges 558, 572. In an example, a thread is provided at a distal portion of the sliding member 554 and a corresponding thread engaging with the thread on the sliding member 554 is arranged at an underside of the thumb rest 538 or as a separate part, for example, as a tubular spacer, between the thumb rest 538 and the sliding member 554. Rotation of the engaging threads relative to each other, for example, by rotating the sliding member 554, the tubular spacer, or the thumb rest 538, respectively, will change a distance by which the sliding member 554 is moved in the direction of the deformable element. In some examples, an actuating member 570 including a pair of engaging threads is further usable for adjusting, by corresponding rotation of the threads, a locking force which is exerted by the locking arrangement 550.

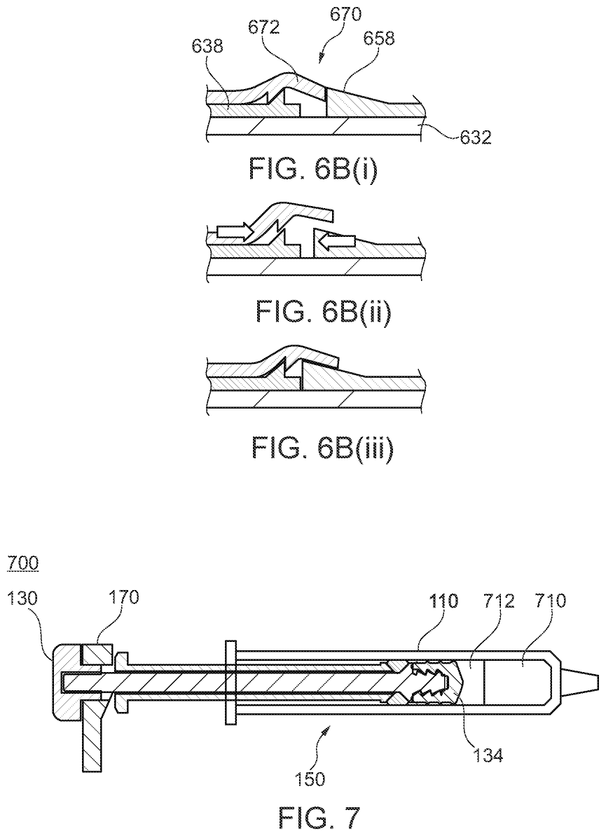

FIG. 6A (which is FIGS. 6A(i), 6A(ii), and 6A(iii)) shows schematically and exemplarily a detail of a locking arrangement 650 according to another example, in a locked state (FIG. 6A(i)), in a released state (FIG. 6A(ii)), and in an unlocked state (FIG. 6A(iii)). What has been described above regarding features and functions of the locking arrangements 150, 350, 450 applies analogously to the locking arrangement 650, unless otherwise clear from FIG. 6 and the following description.

In the locking arrangement 650, the at least one actuating member 670 includes at least one elastic finger 672, which extends from an underside of the thumb rest 638 of the plunger 630 and which engages with a contour 658 at the proximal end of the sliding member 654.

As shown in the upper portion (FIG. 6A(i)) of FIG. 6A, when the locking arrangement is in the locked position, the elastic finger 672 blocks the contour 658 from moving in the outward direction. In this way, the locking arrangement 650 is held in the locked position.

As shown in the middle portion (FIG. 6A(ii)) of FIG. 6A, when a user pulls the sliding member 654 in the outward direction by manual force, the elastic finger 672 will give way and in this manner will release the sliding member 654 from the blocked state.

As shown in the lower portion (FIG. 6A(iii)) of FIG. 6A, once the sliding member 654 has been moved into a position corresponding to an unlocked position of the locking arrangement 650, the elastic finger 672 will engage with the contour 658 such that the sliding member 654 is held back in the outward position. In this way, the locking arrangement 650 is held in the unlocked state.

FIG. 6B (which is FIGS. 6B(i), 6B(ii), and 6B(iii)) shows further details of the locking arrangement 650. As shown in FIG. 6B, a toothed contour is provided in some examples at the underside of the thumb rest 638. In combination with a toothed contour at the elastic finger 672, it can be provided that unlocking the locking arrangement 650 is irreversible.

Instead of pulling the sliding member 654 in the outward direction, in some examples a user may simply push the thumb rest 638 in the inward direction to unlock the locking arrangement 650. As is apparent from the middle portion (ii) in FIG. 6A and/or FIG. 6B, when a user pushes the thumb rest 638 in the inward direction, the plunger 630 may be moved slightly in the inward direction, except for the sliding member 654, which is held back by the deformable element. The slight movement of the plunger 630 relative to the sliding member 654 is enabled by the elastic nature of the elastic finger 672. In turn, the deformation of the elastic finger 672 due to the slight movement of the plunger 630 leads to a releasing of the contour 658 by the elastic finger 672. This operation facilitates a single-handed use of the container and of the locking arrangement 650. In particular, in the case that a user is unaware of the locking arrangement 650 and of its mode of operation, successful application of an injection is still likely, for example, in an emergency situation, since the user is not required to perform any actions different from the use of a conventional syringe, but merely needs to apply an increased pushing force to surmount the resistance exerted by the elastic finger 672.

FIG. 7 shows schematically and exemplarily a container 700 according to another example. Identical reference numerals as in FIG. 1 denote identical features. Furthermore, the above description of FIG. 1 applies correspondingly to the container 700 shown in FIG. 7. A locking arrangement 150 of the container 700 is in the locked position.

The container 700 is pre-filled with a liquid 710. In addition, a gas 712, for example, air, is contained in a headspace of the container 700.

Concerning a motion of the plunger 130, a resultant force acting on the plunger 130 due to thermal expansion and/or contraction of a content of the container 700 depends, among other conditions, on a type and a quantity of the substances 710, 712 contained in the container 700.

In some examples, the deformable element of the locking arrangement 150 is configured to seal the gap between the inner wall of the container body 110 and the plunger 130 against a pressure difference of at least 500 millibars, optionally at least 600 millibars, optionally at least 800 millibars, at a temperature of minus 80 degrees Celsius.

Furthermore, in some examples, the locking arrangement 150 is configured to reduce a motion of the plunger 130 relative to the container body 110 to less than 3 millimeters, optionally less than 2 millimeters, optionally less than 1.5 millimeters, optionally less than 0.5 millimeters, when applying a pressure difference of 500 millibars, optionally 600 millibars, optionally 800 millibars, at a temperature of minus 20 degrees Celsius, optionally minus 50 degrees Celsius, optionally minus 80 degrees Celsius.

The following parameters are fulfilled simultaneously in some examples of the container 700. The brimful volume of the container body 110 is 0.5 to 100 milliliters, optionally 1 to 20 milliliters, optionally 1 to 5 milliliters. In addition, the container 700 is prefilled with the liquid 710 by 10% to 90%, optionally 20% to 80%, optionally 30% to 70%, optionally 40% to 60% of the brimful volume of the container 700. Optionally, the liquid 710 includes a sugar or salt. In addition, the container (700) includes a headspace of gas (712), optionally air, of 0 to 10 millimeters, optionally 0.5 to 5 millimeters, optionally 1 to 4 millimeters, optionally 1.5 to 3 millimeters. In addition, a movement of the plunger rod is 3 millimeters or less, optionally 2 millimeters or less, optionally 1 millimeter or less, optionally 0.5 millimeters or less, optionally 0 millimeters, after one or both of the following test conditions were applied.

Test Condition 1: The pressure outside the container 700 with regard to the inside was reduced by 100 to 1000 millibars, optionally 200 to 900 millibars, optionally 300 to 800 millibars, more optionally 400 to 600 millibars, optionally 430 millibars for 1 second to 1 week, optionally 1 minute to 24 hours, optionally 1 hour to 6 hours, optionally 180 minutes.

Test Condition 2: The container 700 was stored at −150° C. to 25° C., optionally −120° C. to 0° C., optionally −100° C. to −15° C., optionally −90° C. to −40° C., optionally −80° C., for 1 second to 7 days, optionally 1 minute to 24 hours, optionally 1 hour to 6 hours, optionally 180 minutes.

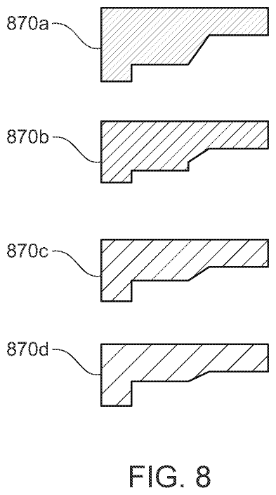

FIG. 8 shows a set of actuating members 870a to 870d. The actuating member 870a to 870d are usable interchangeably, for example, to serve as the actuating member 170, in the form of a pin, in connection with the locking arrangement 150 shown in FIGS. 1 and 7. As can be seen in FIG. 8, the actuating members 870a to 870d differ from each other with respect to a thickness of their wedged profile. Accordingly, by selecting a respective one among the actuating members 870a to 870d, a resulting locking force of the locking arrangement can be adjusted. In an example, a total break-loose and gliding force of a plunger as part of a container of the described type is adjusted by corresponding choice of one of the actuating members 870a to 870d.

In the foregoing, examples of locking arrangements have been described in particular connection with a syringe. However, as will be appreciated, some or all of the described features and functions of a locking arrangement can be implemented correspondingly in connection with other types of containers, such as cartridges.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A syringe, comprising:
a container body including an inner wall;
a plunger including a plunger rod, the plunger rod being disposed at least partially within the container body; and
a locking arrangement configured for locking a position of the plunger rod relative to the container body and for being operable between at least a locked position and an unlocked position, the locking arrangement including at least one deformable element which is configured for applying a locking force between the plunger rod and the inner wall of the container body when the locking arrangement is in the locked position and for releasing the locking force between the plunger rod and the inner wall of the container body when the locking arrangement is changed from the locked position to the unlocked position.

2. The syringe according to claim 1, wherein the locking arrangement is attached to the plunger rod.

3. The syringe according to claim 1, wherein the container body includes an inner periphery, the at least one deformable element being arranged to extend along the inner periphery of the container body.

4. The syringe according to claim 3, wherein (i) the inner periphery of the container body is essentially circular, and the at least one deformable element has an essentially circular shape, or (ii) the inner periphery of the container body is essentially polygonal, and the at least one deformable element has an essentially polygonal shape.

5. The syringe according to claim 1, wherein the at least one deformable element is further configured to seal a gap between the inner wall of the container body and the plunger rod when the locking arrangement is in the locked position.

6. The syringe according to claim 5, wherein the at least one deformable element is configured to seal the gap between the inner wall of the container body and the plunger rod against a pressure difference of at least 500 Millibars, at a temperature of minus 80 degrees Celsius.

7. The syringe according to claim 6, wherein the pressure difference is at least 600 Millibars or at least 800 Millibars.

8. The syringe according to claim 1, wherein the locking arrangement further comprises a lateral protuberance which extends from a portion of the plunger rod, and wherein the at least one deformable element is arranged to be pressed towards the lateral protuberance when the locking arrangement is in the locked position.

9. The syringe according to claim 8, wherein the locking arrangement further comprises a sliding member which is configured for moving relative to the plunger rod and is configured to apply a pressing force on the at least one deformable element towards the lateral protuberance when the locking arrangement is in the locked position.

10. The syringe according to claim 9, wherein at least one of the lateral protuberance and the sliding member are configured to cause at least one of deformation and displacement of at least a portion of the at least one deformable element towards the inner wall of the container body when the locking arrangement is in the locked position.

11. The syringe according to claim 9, wherein at least one of:
the lateral protuberance extends from the plunger rod, and the sliding member comprises a tube in which the plunger rod extends at least partially; and
the locking arrangement further comprises at least one actuating member configured for at least one of (a) moving the sliding member towards the at least one deformable element when bringing the locking arrangement to the locked position, and (b) for releasably holding the locking arrangement in the locked position.

12. The syringe according to claim 1, wherein at least one of:
(a) the container body is uncoated or silicone-free coated in an area of the container body contacting with the at least one deformable element when the locking arrangement is in the locked position; and
(b) at least one of (i) a material of the at least one deformable element comprises a silicone, and (ii) the at least one deformable element is silicone oil-free coated.

13. The syringe according to claim 12, wherein the at least one deformable element is silicone oil-free coated, liquid-free coated, or uncoated.

14. The syringe according to claim 1, wherein the container is pre-filled.

15. The syringe according to claim 1, wherein the locking arrangement is configured to reduce a motion of the plunger relative to the container body to less than 3 millimeters, when applying a pressure difference of 500 Millibars, at a temperature of minus 20 degrees Celsius.

16. The syringe according to claim 1, wherein the locking arrangement is configured to apply the locking force in a range of 5 to 15 newtons.

17. The syringe according to claim 1, wherein at least one of:
(a) a contact length between the at least one deformable element and the inner wall of the container body is in a range of 0 to 1 millimeters, when the locking arrangement is in the unlocked position; and
(b) a contact length between the at least one deformable element and the inner wall of the container body is in a range of 2 to 20 millimeters, when the locking arrangement is in the locked position.

18. The syringe according to claim 1, further comprising a stopper, wherein the at least one deformable element is not in direct contact with the stopper.

19. The syringe according to claim 1, wherein the locking arrangement is in the locked position and the following parameters are fulfilled:
a brimful volume of the container body is 0.5 to 100 milliliters;
the container is configured for being prefilled with a liquid;
the container further comprises a headspace of gas of 0 to 40 millimeters;
wherein a movement of the plunger rod is 3 millimeters or less, after at least one of the following test conditions has been applied:
(i) a pressure outside the container with regard to an inside of the container is reduced by 100 to 1000 millibars, for 1 second to 1 week;
(ii) the container is stored at −210° C. to 25° C., for 1 second to 7 days.

20. The syringe according to claim 19, wherein the locking arrangement is in the locked position and the following parameters are fulfilled:
the brimful volume of the container body is 1 to 20 milliliters or 1 to 5 milliliters;
the container is configured for being prefilled with the liquid, with 10% to 90%, 20% to 80%, 30% to 70%, or 40% to 60% of the brimful volume of the container, wherein the liquid comprises a sugar or a salt;
the container further comprises the headspace of air of 0 to 40 millimeters, 0.5 to 5 millimeters, 1 to 4 millimeters, or 1.5 to 3 millimeters;
wherein a movement of the plunger rod is 2 millimeters or less, 1 millimeter or less, 0.5 millimeters or less, or 0 millimeters, after at least one of the following test conditions has been applied:
(i) the pressure outside the container with regard to the inside of the container is reduced by 200 to 900 millibars, 300 to 800 millibars, 400 to 600 millibars, or 430 millibars for 1 second to 1 week, 1 minute to 24 hours, 1 hour to 6 hours, or 180 minutes;
(ii) the container is stored at −120° C. to 0° C., −100° C. to −15° C., −90° C. to −40° C., or −80° C., for 1 second to 7 days, 1 minute to 24 hours, 1 hour to 6 hours, or 180 minutes.
